(19) United States
(12) Patent Application Publication
(10) Pub. No.: US 2021/0231685 A1
(43) Pub. Date: Jul. 29, 2021

(54) VITAMIN D BINDING PROTEIN IN THE CLINICAL MANAGEMENT OF HEMATOPOIETIC STEM CELL TRANSPLANTATION

(71) Applicant: CHILDREN'S HOSPITAL MEDICAL CENTER, Cincinnati, OH (US)

(72) Inventors: Stella M. Davies, Cincinnati, OH (US); Nathan Luebbering, Cincinnati, OH (US)

(21) Appl. No.: 17/250,025

(22) PCT Filed: May 7, 2019
(86) PCT No.: PCT/US2019/031053

§ 371 Date: Nov. 6, 2020

Related U.S. Application Data
(60) Provisional application No. 62/820,428, filed on Mar. 19, 2019.
(60) Provisional application No. 62/667,664, filed on May 7, 2018.

(51) Int. Cl. G01N 33/68; A61K 38/17; A61P 37/06; G01N 33/82

(52) U.S. Cl. CPC G01N 33/6893; A61K 38/1722; G01N 2800/52; G01N 33/82; G01N 2800/245; A61P 37/06

(57) ABSTRACT

The present invention relates to methods for improving the clinical management of hematopoietic stem cell transplant (HSCT) recipients by identifying patients at increased risk for non-relapse mortality or a transplantation associated adverse event using serum or plasma levels of vitamin D binding protein (VDBP) as a biomarker and related methods of monitoring therapy using same and the use of VDBP in therapy to prevent non-relapse mortality or a transplantation associated adverse event in a HSCT recipient, especially recipients identified as being at high risk.

### STATEMENT REGARDING FEDERALLY-SPONSORED RESEARCH

This invention was made with U.S. Government support under R01 HD 093773 awarded by the National Institutes of Health. The U.S. Government has certain rights in the invention.

### TECHNICAL FIELD

The present invention relates to the field of medicine, and more particularly to the field of hematopoietic stem cell transplant (HSCT), especially in the context of the clinical management of patient recovery following HSCT.

### BACKGROUND

Chemotherapy and radiation are used in very high doses to purposely eradicate an entire organ, the hematopoietic system, in situ, inside a human body prior to hematopoietic stem cell transplant (HSCT). This is a bold endeavor, and significant toxicity results, most notably in the first four weeks after transplantation, when morbidity and mortality are high. Biomarker data support the hypothesis that early events, in the first 14 days after transplant, set the scene for later adverse events such as transplant-associated thrombotic microangiopathy (TA-TMA) and graft versus host disease (GVHD) which occur 21-60 days after HSCT (Rotz et al., N Engl J Med, 2017. 376(12):1189-1190).

Endothelial injury seems to be a key event in the initiation of TA-TMA and GVHD, and contributes significantly to organ injury and death (Gloude et al., Blood, 2017. 130(10) 1259-1266; Jodele et al., Blood Rev, 2015. 29(3):191-204; Tichelli and Gratwohl, Best Pract Res Clin Haematol, 2008. 21(2):139-48).

Cell lysis leads to release of toxic intracellular molecules into the circulation, including F-actin and nucleotides such as ATP, that are usually present in low concentrations outside the cell. Actin is the most abundant protein in the human body and exists in two forms—monomeric or globular actin (G-actin) and a polymeric form, filamentous or F-actin. F-actin is known to be angiopathic, and an effective actin scavenger system exists to rapidly clear F-actin that enters the circulation. Extracellular F-actin is depolymerized into G-actin by plasma gelsolin. Monomeric G-actin then binds to the highly abundant circulating protein, vitamin D binding protein (VDBP), and the actin-VDBP complex is removed from the circulation by the reticulo-endothelial system, primarily in the liver (Lind et al., J Clin Invest, 1986. 78(3):736-42). VDBP has been shown to be consumed in other clinical circumstances of significant cell lysis such as acute liver failure and major trauma.

VDBP is an abundant protein, related to albumin and alpha-fetoprotein. VDBP is conserved of throughout evolution, and no humans deficient in VDBP have been identified, indicating important biological function for VDBP. VDBP is a highly polymorphic molecule, with more than 70 different alleles described, and the frequency of three major alleles, Gc1s, Gc1f and Gc2 varies by race. VDBP transports the major circulating form of vitamin D, 25-hydroxyvitamin D, and other vitamin D metabolites in the circulation, but only about 4% of VDBP is conjugated to vitamin D metabolites, indicating other important functions for the molecule. A deglycosylated form of VDBP has been shown to have immunomodulatory activity, serving as a so-called “macrophage-activating factor” (Yamamoto and Naraparaju, Cell Immunol, 1996. 170(2):161-7).

### SUMMARY

The present invention is based, in part, on the discovery that serum F-actin is a significant contributor to endothelial cell injury following hematopoietic stem cell transplant (HSCT) and that vitamin D binding protein (VDBP) plays an important role in reducing serum F-actin and is a biomarker for clinical outcome in HSCT recipients. These discoveries are translated as described herein into methods for identifying subjects at increased risk of non-relapse mortality and transplantation related adverse events, methods of treating HSCT recipients with anti-F actin and VDBP therapies, and methods of monitoring the efficacy of such treatments in order to improve clinical outcomes for HSCT patients.

Accordingly, the disclosure provides methods for identifying a hematopoietic stem cell transplant (HSCT) recipient at increased risk for post-HSCT non-relapse mortality or a transplantation associated adverse event, the method comprising determining ex vivo or in vitro a level of vitamin D binding protein (VDBP) in a serum or plasma sample obtained from the HSCT recipient, wherein a level of VDBP below a predetermined threshold identifies the HSCT recipient as being at increased risk of non-relapse mortality or a transplantation associated adverse event. In embodiments, the methods comprise determining ex vivo or in vitro the level of one or more additional proteins in the serum or plasma sample of the HSCT recipient, the one or more additional proteins selected from F actin, IL-6, IL-10, and TNFα. In embodiments, the methods comprise determining the level of VDBP or the level of the one or more additional proteins in the serum or plasma sample comprises an immunoassay, optionally an enzyme linked immunosorbent assay (ELISA).

In embodiments, the transplant associated adverse event is selected from transplant-associated thrombotic microangiopathy (TA-TMA) and graft versus host disease (GVHD).

In embodiments, the disclosure also provides methods for monitoring a therapy of an HSCT recipient, the method comprising determining ex vivo or in vitro a level of VDBP in a serum or plasma sample obtained from the HSCT recipient, wherein a level of VDBP below a predetermined threshold indicates that the recipient is at increased risk of non-relapse mortality or a transplantation associated adverse event and that the therapy should be adjusted. In embodiments, the methods comprise determining the level of VDBP at multiple time points during the therapy. In embodiments, the methods comprise determining whether the level of VDBP is increasing or decreasing over time.

In embodiments, the disclosure also provides methods for treating an HSCT recipient, the methods comprising administering purified or recombinant human VDBP to the recipient following transplantation, for example by intravenous infusion during the first 1-2 weeks or during the first 1-3 months following transplanation. In some embodiments the purified or recombinant human VDBP may be administered during the first 1-6 months or 1-12 months following transplantation.

In certain embodiments of any of the methods described here, the recipient is human. In embodiments, the human HSCT recipient has been diagnosed with a lymphoma, leukemia, immune-deficiency disease or disorder, a congenital metabolic defect, a hemoglobinopathy, a myelodysplastic syndrome, or a myeloproliferative syndrome. In embodiments, the human HSCT recipient has been diagnosed with a hematological malignancy or premalignant condition. In embodiments, the hematological malignancy or premalignant condition is selected from acute myeloid leukemia (AML), acute lymphoblastic leukemia, chronic myeloid leukemia, Hodgkin lymphoma (HL), non-Hodgkin lymphoma (NHL), and multiple myeloma.

The disclosure also provides the use of purified or recombinant human VDBP in a method of therapy to prevent or reduce the risk of non-relapse mortality or a transplantation associated adverse event in a HSCT recipient, preferably a human HSCT recipient. In embodiments, the HSCT recipient is identified as being at high risk for non-relapse mortality or a transplantation associated adverse event. In embodiments, the VDBP is purified from human plasma or serum. In embodiments, the VDBP is purified using a method comprising high performance liquid chromatography or affinity chromatography.

### BRIEF DESCRIPTION OF THE DRAWINGS

FIG. 1A-D: Relationship of F actin and VDBP serum levels to cumulative incidence of adverse events in HSCT recipients. A, Cumulative incidence of TA-TMA in HSCT recipients with (dotted line) and without (solid line) detectable F-actin (61% vs 37%, p=0.03), measured from zero to 350 days after translplantation. B, Cumulative incidence of non-relapse mortality (NRM) in HSCT recipients with (dotted line) and without (solid line) detectable F-actin (29% vs 13%, p=0.028), measured from zero to 350 days after translplantation. C, Cumulative incidence of TA-TMA in HSCT recipients with VDBP levels above (solid line) and below (dotted line) the median (10% vs 31%, p=0.01), during the time from zero to 5 years after transplanation. D, Cumulative incidence of NRM in HSCT recipients with VDBP levels above (solid line) and below (dotted line) the median (0% vs 15%, p=0.002), during the time from zero to 5 years after transplanation.

FIG. 2A-C: VDBP serum levels following HSCT. A, Western blot probed for VDBP showing serum collected pre-transplant, and at days 0, 7, and 14 after transplant from two HSCT recipients. A lower molecular weight band corresponding to VDBP (unbound to F-actin) is seen in all samples including pre-transplant serum (BL). However, in post-transplantation serum a larger slower migrating band was also observed corresponding to VDBP bound to actin collected on days 0,7 and 14, demonstrating presence of circulating actin-VDBP complex in samples from UPN 198. Similar increase in levels of circulating actin-VDBP complex is not seen in UPN 436, illustrating inter-individual variability. BL=baseline, sample collected before the start of transplant. B, Western blot probed for VDBP showing clearance of increased actin-VDBP complexes from the circulation, with complexes largely cleared by days 21-28 after HSCT in two different transplant recipients (UPN 324 and UPN 336). C, VDBP levels according to genotype, measured at baseline, days 0, 7, 14, 30 and 100 after HSCT, showing decreased levels at day 14 in all genotypes. (●) Gc1S/Gc1S, (▪) Gc1S/Gc1F, (▴) Gc1S/Gc2, (▾) Gc1F/Gc1F, (♦) Gc1F/Gc2, ( [symbol] ) Gc2/Gc2.

FIG. 3. Extracellular acidification rate (ECAR) in peripheral blood mononuclear cells obtain from normal donor and incubated with serum collected from two different HSCT transplant recipients. Top panel: Cells were incubated with serum from HSCT transplant recipient who exhibited increasing levels of VDBP from a baseline VDBP level of 78 mg/ml to a day 100 VDBP level of 907 mg/ml (genotype Gc1F/Gc1F); top line is Day 100 and bottom (darker) line is Baseline. Lower panel: Cells were incubated with serum from a second HSCT transplant recipient in whom VDBP levels were more constant (baseline VDBP level 681 mg/ml, Day 100 level 727 mg/ml, genotype Gc1S/Gc2); darker line is Day 100 and lighter line is Baseline.

FIG. 4: Schematic of proposed VDBP activity during HSCT, with predominant effect early after transplant being actin scavenging with later effects on macrophage polarization and tissue healing

FIG. 5: ATP levels measured by mass spectrometry in samples collected from four HSCT recipients prior to BMT and at time of ANC nadir (maximum cell lysis). The bars demonstrate area of peak intensity from the SRM transition of each nucleoside phosphate as denoted in the methods section.

### DETAILED DESCRIPTION

The disclosure provides methods for identifying hematopoietic stem cell transplantation (HSCT) recipients who are at increased risk of non-relapse mortality and transplantation related adverse events, as well as methods of treating HSCT recipients with anti-F actin and VDBP therapies, and methods of monitoring the efficacy of such treatments in order to improve clinical outcomes for HSCT patients.

In accordance with the methods described here, an HSCT recipient is a patient in need of reestablishment of hematopoietic function, for example due to disease, damage, defects in their bone marrow or immune system. In accordance with the methods described here, a subject in need is preferably a human subject who is in need of a hematopoietic stem cell transplant (HSCT), or who has received such a transplant, for example in connection with a treatment regimen for a disease or disorder such as a lymphoma, leukemia, immune-deficiency disease or disorder, a congenital metabolic defect, a hemoglobinopathy, a myelodysplastic syndrome, or a myeloproliferative syndrome. In some embodiments, the HSCT recipient is a patient diagnosed with a lymphoma, leukemia, immune-deficiency disease or disorder, a congenital metabolic defect, a hemoglobinopathy, a myelodysplastic syndrome, or a myeloproliferative syndrome. In some embodiments, the HSCT recipient is a patient diagnosed with a non-malignant disease or disorder. In embodiments, the non-malignant disease or disorder may be selected from an anemia, sickle cell disease, thalassemia, hemoglobinuria, Chediak-Higashi syndrome, chronic granulomatous disease, Glanzmann thrombasthenia, osteopetrosis, a lysosomal storage disorder such as Gaucher disease and Niemann-Pick disease, mucopolysaccharidosis, glycoproteinoses, ataxia telangiectasia, DiGeorge syndrome, Severe combined immunodeficiency (SCID), Wiscott-Aldrich disease, Kostmann syndrome, and Shwachman-Diamond syndrome. In some embodiments, the HSCT recipient is a patient diagnosed with a hematological malignancy or premalignant condition. In embodiments, the hematological malignancy or premalignant condition is selected from acute myeloid leukemia (AML), acute lymphoblastic leukemia, chronic myeloid leukemia, Hodgkin lymphoma (HL), non-Hodgkin lymphoma (NHL), and multiple myeloma.

Typically, the HSCT recipient has received, prior to transplantation, intensive myeloablative chemoradiotherapy, which may take place over a period of days, for example 7-10 days. Following myeloablative chemoradiotherapy, the patient receives an infusion of either autologous hematopoietic stem cells or allogeneic human leukocyte antigen (HLA)-matched hematopoietic stem cells from a donor. In accordance with the methods described here, ‘day 0’ or the zero time point in the methods comprising assaying for a biomarker at multiple time points, refers to the day before initiation of myeloablative chemoradiotherapy. Subsequent time points, e.g., day 7, day 10, day 14, day 28, refer to days after the HSCT recipient has received autologous or allogeneic hematopoietic stem cells.

In some embodiments, the methods described here comprise assaying a serum or plasma sample obtained from an HSCT recipient to determine the level of a risk associated biomarker, vitamin D binding protein (VDBP), the risk being selected from non-relapse mortality and a transplantation associated adverse event, for example transplant-associated thrombotic microangiopathy (TA-TMA) and graft versus host disease (GVHD). In accordance with the methods described here, biomarker levels may be measured in either a plasma or serum sample obtained from the subject. In embodiments, the assaying step comprises determining the levels of VDBP in the serum or plasma sample using an immunoassay, for example an enzyme linked immunosorbent assay (ELISA). In embodiments, the methods may further comprise a step of comparing the VDBP level to a reference VDBP level, to determine whether the HSCT recipient's serum VDBP level is above or below the reference VDBP level. In embodiments, the assaying step may be performed more than once, for example on two or more serum or plasma samples obtained from the HSCT recipient at two or more time points following transplantation and/or following initiation of a treatment or therapy. For example, VDBP serum levels may be assayed at two or more time points including time zero (0) which corresponds to the day before initiation of myeloablative chemoradiotherapy, and 7-14 or 7-28 days post-transplantation. In some embodiments, VDBP serum levels are assayed at regular intervals post-transplantation, for example weekly or bi-weekly for a period of months, for example 2-6 months or 6-12 months, or for a period of 1-2 years, in order to monitor the HSCT recipient's response to therapy or changing risk profile.

In embodiments, the methods may further comprising assaying at least one additional serum biomarker. In embodiments, the at least one additional serum biomarker is selected from one or more of F actin, interleukin 6 (IL-6), interleukin 10 (IL-10), and tumor necrosis factor alpha (TNFα).

The disclosure also provides methods of treating an HSCT recipient with a therapy selected from anti-F actin therapy and VDBP therapy, and combinations thereof. In embodiments, the anti-F actin therapy is an immunotherapy, for example an antibody against F actin. In embodiments, the VDBP therapy comprises administering exogenous VDBP to the patient. Exogenous VDBP may be obtained, for example, by purification of VDBP from pooled serum or via the production of recombinant VDBP. In embodiments, VDBP may be purified from human serum using a chromatographic method, for example a method comprising high pressure liquid chromatography (HPLC). Such a method was exemplified by Taylor et al. in Clin Chim Acta 1986 155:31-41. In embodiments, VDBP may be purified from human serum using an affinity chromatography method, for example as described in Samy et al., Protein Expr Purif 1995 6:185-188.

In embodiments, VDBP therapy comprises administering VDBP to the HSCT recipient at one or more time points after transplantation, for example during the first 7-14 days after transplantation, or during the first 1-4 months or 1-3 months after transplantation, for example by intravenous infusion of purified or recombinant VDBP. In embodiments, infusion of VDBP may occur once daily, two or three times a week, once weekly, biweekly, or with such other frequency as warranted by the patient's clinical presentation and/or risk profile for non-relapse mortality and transplantation related adverse events.

In embodiments, anti-F actin and/or VDBP therapy is accompanied by monitoring the patient's serum F actin levels or serum VDBP levels, or both, for example at regular intervals during therapy using an immunoassay as described herein.

In accordance with the methods described here, a biological serum sample is obtained from a subject in need, wherein the subject is a HSCT recipient, or intended HSCT recipient, and assayed for the presence of a risk biomarker. The serum sample may be obtained from a sample of whole blood obtained from the subject. The term “whole blood” refers to a sample of blood containing cells, such as red blood cells (also referred to as erythrocytes), white blood cells, and platelets, as well as plasma, which is the liquid remaining after the cells are removed. In some embodiments, the methods may comprise a step of obtaining whole blood from a subject, e.g., by phlebotomy. The blood collected is preferably venous blood which may be collected into a suitable container, e.g., a container comprising sodium heparin.

In embodiments, the methods described here comprise determining whether the amount of an analyte, i.e., VDBP, is above or below a predetermined threshold, wherein the amount relative to the predetermined threshold indicates whether the subject is at increased risk for non-relapse mortality (NRM) or a transplantation associated adverse event, such as transplant-associated thrombotic microangiopathy (TA-TMA) or graft versus host disease (GVHD), or both. The diagnostic threshold for the analyte is pre-determined based on the amount of the analyte in a representative population of healthy disease-free donor subjects. In embodiments, the performance of the method is measured using an area under the curve (AUC) receiver operating characteristics (ROC) curve.

The methods of the present disclosure are preferably applicable to human subjects, also referred to as “patients”, but the methods may also be applied to other mammalian subjects. Accordingly, in embodiments a method described here may be performed on a “subject” which may include any mammal, for example a human, primate, mouse, rat, dog, cat, cow, horse, goat, camel, sheep or a pig. Preferably, the subject is a human. The term “patient” refers to a human subject.

Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3^{rd} ed., Cold Spring Harbor Laboratory Press (Cold Spring Harbor, N.Y. 2001) provide one skilled in the art with a general guide to many of the terms used in the present application.

All percentages and ratios used herein, unless otherwise indicated, are by weight. Other features and advantages of the present disclosure are apparent from the different examples set forth below. The examples illustrate different components and methodologies useful in practicing aspects of the present disclosure. The examples do not limit the claimed disclosure. Based on the present disclosure the skilled artisan can identify and employ other components and methodologies useful for practicing the methods described here.

### EXAMPLES

Our initial aim was to identify mechanisms of endothelial injury, and to identify potential factors that ameliorate injury and modify individual risk of TA-TMA and GVHD. We hypothesized that endothelial injury after HSCT is due, at least in part, to release of F-actin, and that higher levels of VDBP would protect against endothelial injury and be associated with improved outcomes after transplant. Our data indicate a significant and beneficial role for VDBP in HSCT which appears to involve both actin scavenging and modulation of macrophage phenotype. In sum, and as discussed in more detail below, we detected F-actin in the blood of 52% of HSCT recipients in the first 14 days after HSCT, and recipients with detectable F-actin had significantly elevated risk of TA-TMA (p=0.03) and non-relapse mortality (NRM) (p=0.04). In a cohort of 190 children receiving allogeneic HSCT risk of TA-TMA was reduced in those with serum concentrations of VDBP above the median at day 30 (10% vs 31%, p=0.01), and that GVHD and NRM were reduced in those with levels above the median at day 100 (3% vs 18%, p=0.04 and 0% vs 15%, p=0.002). Western blot analyses demonstrated actin-VDBP complexes in the blood, which cleared by around day 21-28. Our data also indicate the modulation of cytokine secretion and macrophage phenotype by VDBP at later times after transplant. Taken together, our data identify F-actin as a mediator of endothelial damage and VDBP as a modifier of individual risk of clinical adverse events consequent to endothelial injury, including mortality, TA-TMA and GVHD.

### Circulating F-Actin Determines Clinical Outcomes of HSCT

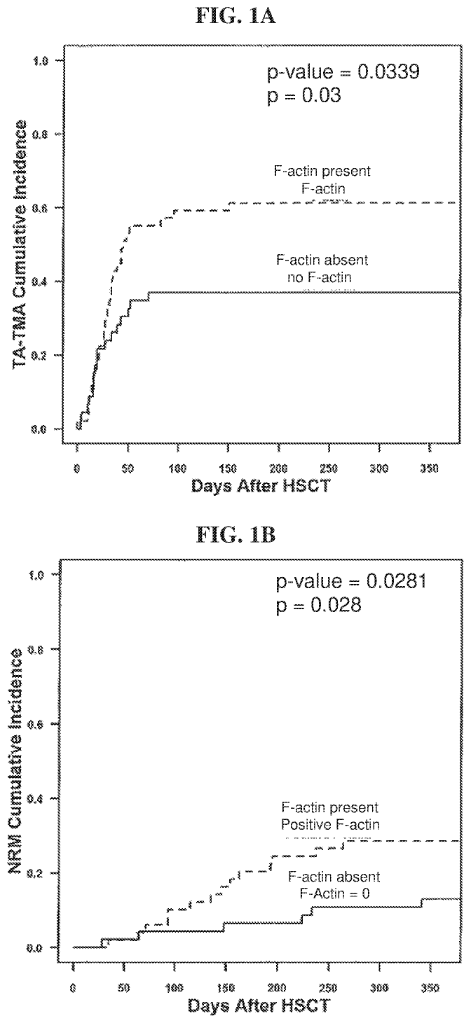

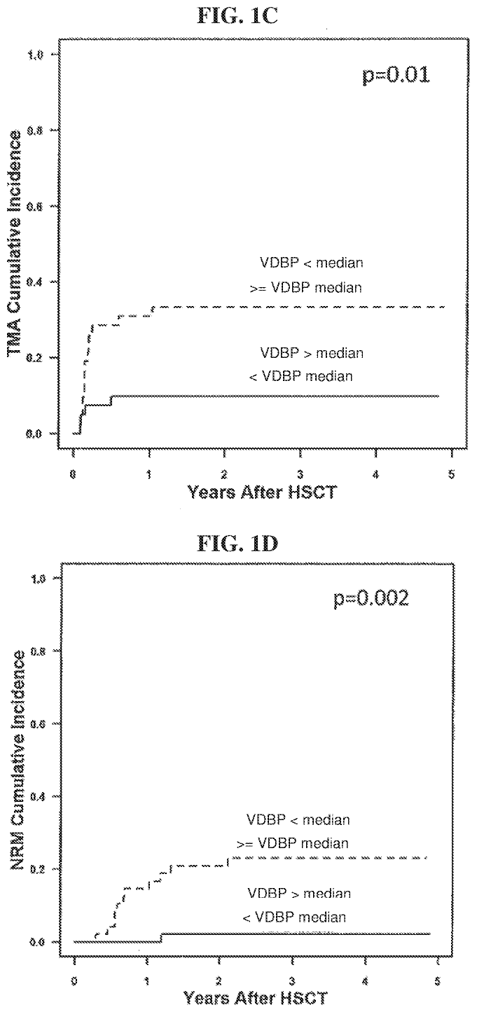

Our overall goal was to understand the mechanism of endothelial injury after HSCT. We hypothesized that F-actin is released into the circulation during lysis of hematopoietic cells caused by chemotherapy and radiation given prior to HSCT. F-actin is known to be toxic to endothelial cells and is generally not present in the circulation. First we looked for detectable F-actin in the circulation at 3 time points after HSCT (baseline, days 0, 7, 14) in an unselected cohort of 96 consecutive transplant recipients who consented to participate in our institutional HSCT sample repository and scored any finding of F-actin as positive. Cases with no F-actin measured at any of the three time points were scored as negative. 50 of 96 (52%) cases analyzed had detectable F-actin. We then examined the impact of F-actin presence in the first 14 days of transplant on outcomes of transplant. We found higher risk of TA-TMA in those with F-actin in the circulation (61% vs 37% at 1 year, p=0.03, FIG. 1A). Moreover, NRM was increased in those with F-actin in the circulation (29% vs 13%, p=0.028, FIG. 1B).

### Circulating Concentrations of VDBP and VDBP Genotype Determine Clinical Outcomes

VDBP is known to function as an actin scavenger, removing angiopathic F-actin from the circulation. We measured VBDP levels at 6 time points after HSCT (baseline, days 0, 7, 14, 30 and 100) in an expanded cohort of 190 consecutive HSCT recipients. Demographics of the cohort are shown in Table 1.

TABLE 1
Demographics of patients included in assessment
of clinical outcomes.

| Characteristic | N = 190 | |
|---|---|---|
| Gender | | |
| Male | 109 | (57%) |
| Female | 81 | (43%) |
| Mean age, yr (range) | 9.8 (0.2-34.7) | |
| Diagnosis | | |
| Bone Marrow Failure | 54 | (28%) |
| Immune Deficiency | 54 | (28%) |
| Malignancy | 42 | (22%) |
| Non-malignant Hematology | 32 | (17%) |
| Genetic/metabolic | 8 | (5%) |
| Race | | |
| White | 163 | (86%) |
| Nonwhite | 27 | (14%) |
| Conditioning regimen | | |
| Myeloablative | 124 | (65%) |
| Reduced Intensity | 66 | (35%) |
| Donor type | | |
| Related | 59 | (31%) |
| Unrelated | 131 | (69%) |
| Stem cell source | | |
| Bone marrow | 127 | (67%) |
| Peripheral blood stem cell | 52 | (27%) |
| Umbilical cord blood | 11 | (6%) |
| HLA match | | |
| 6/6 or 8/8 Allele matched | 142 | (75%) |
| Mismatched | 48 | (25%) |

VDBP levels prior to start of transplant, and at day 0 and day 7 had no impact on transplant outcomes. In contrast, a higher day 30 VDBP level was associated with lower occurrence of TA-TMA in the first year after HSCT (31% vs 10%, p=0.01, FIG. 1C, Table 2). The rate of GVHD at one year was also higher in those with a lower VDBP level, although this did not reach statistical significance (21% vs 8%, p=0.09, Table 2). We then analyzed VDBP level at day 100, looking for associations with clinical events occurring between day 100 and 1 year (summarized in Table 2.) NRM at one year was significantly higher in HSCT recipients with a VDBP level lower than the median at day 100 (15% vs 0%, p=0.002, Table 2, FIG. 1D) Moreover, GVHD at one year was more frequent in HSCT recipients with a VDBP level lower than the median at day 100 (18% vs 3%, p=0.04, Table 2). In contrast, rates of TA-TMA did not correlate with VDBP level at day 100.

TABLE 2
Outcomes of BMT in pediatric HSCT recipients according
to VDBP level at days 30 and 100.

| | Day 30 VDBP | | | Day 100 VDBP | | |
|---|---|---|---|---|---|---|
| | DBP > | DBP < | | DBP > | DBP < | |
| Outcome | Median | Median | p-value | Median | Median | p-value |
| TA-TMA | 10% | 31% | 0.01 | 0% | 7% | 0.45 |
| GVHD | 8% | 21% | 0.09 | 3% | 18% | 0.04 |
| NRM | 8% | 10% | 0.1 | 0% | 15% | 0.002 |

VDBP is a highly polymorphic protein, with three major alleles (Gc1F, Gc1S and Gc2) and a large number of rare minor alleles. We asked whether VDBP genotype influenced transplant outcomes by comparing transplant outcomes in HSCT recipients with the three homozygous genotypes Gc1F/Gc1F, Gc1S/Gc1S and Gc2/Gc2 (Table 3). Genotyping was performed using host DNA stored prior to HSCT as VDBP is primarily synthesized in the liver. We analyzed homozygous cases only to maximize our chance of seeing biological differences related to genotype (90 total cases, 57 Gc1s/Gc1S, 16 Gc1F/Gc1F and 17 Gc2/Gc2).

TABLE 3
Outcomes of HSCT according to host VDBP genotype,
in those with a homozygous genotype.

| | Number | | | | |
|---|---|---|---|---|---|
| | of cases | TA-TMA | p-value | NRM | p-value |
| Gc1S/Gc1S | 57 | 33% | | 12% | |
| Gc1F/Gc1F | 16 | 63% | | 13% | |
| Gc2/Gc2 | 17 | 6% | | 0% | |
| | | | 0.001 | | 0.22 |

The data show a significant difference in frequency of TA-TMA according to genotype with the highest frequency seen in Gc1F/Gc1F individuals and the lowest in those with a Gc2/Gc2 genotype. NRM frequencies were not statistically different.

### Actin-VDBP Complex Formation in HSCT Patients

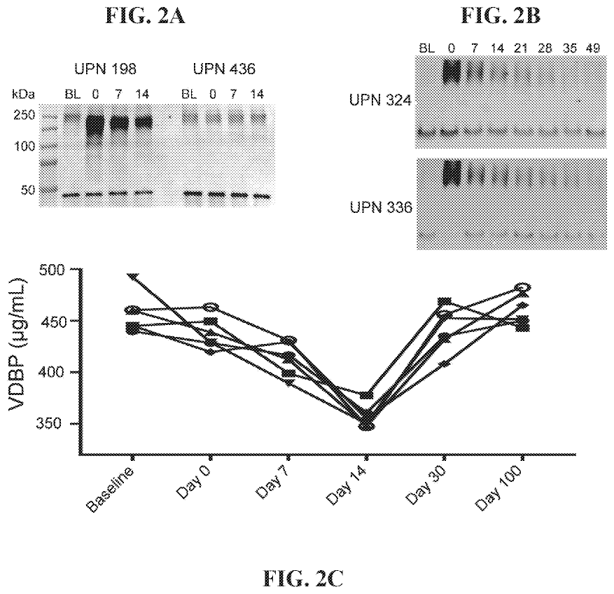

We next investigated the kinetics of VDBP-actin complexes in the circulation. Typically, F-actin is cleared rapidly from the circulation by depolymerization by gelsolin, followed by binding of monomeric actin to VDBP. The actin-VDBP complex is then removed from the circulation by the reticulo-endothelial system with a reported half-life of 30 minutes. We first examined actin-VDBP complex formation by Western blot analysis of serum VDBP after running a native gel to identify mobility changes of serum VDBP due to binding of actin released by cell lysis. Results for analysis of serum VDBP from one patient (UPN198) showed the presence of lower molecular weight band corresponding to VDBP (unbound to F-actin) in all samples including pre-transplant serum (BL). However, in post-transplantation serum a larger slower migrating band was also observed corresponding to VDBP bound to actin (FIG. 2A). There was variability in the presence of complex seen after transplant, with large amounts of complex seen in some cases, while less or none was seen in others. For example, UPN 198, had a large amount of complex present post-transplant, while a second patient, UPN 436 had modest and similar amounts of complex present at all time points examined (FIG. 2A).

We considered whether the presence of a large amount of complex on day 0 might be associated with inferior outcomes, as has been suggested by a prior animal study. Western blot analysis of VDBP was carried out using serum from 93 transplant recipients, and each blot scored as positive (as seen in UPN 198, FIG. 2A, left panel) or negative (as seen in UPN 436, FIG. 2A right panel) for the presence of elevated VDBP-actin complex. We found no association between the presence of elevated VDBP-actin complex and TA-TMA, (35% vs 40%, p=0.67), GVHD (35% vs 26%, p=0.39) or NRM (5% vs 10%, p=0.66). We also considered whether the amount of complex simply reflected the level of VDBP but found no such association (p=0.17).

VDBP bound to F-actin is removed from the circulation by the reticuloendothelial system, so that serum levels of VDBP-actin complex are likely to change post-transplant. We therefore examined the kinetics of clearance of complex in a patient with a large amount of complex further by examining later time points after infusion of stem cells (FIG. 2B). Data show that levels of actin-VDBP complex returned to baseline by 21-28 days after transplant. Further analysis showed a temporal decline in serum DBP levels at day 14 post-transplantation, common to all patients and all VDBP genotypes, with a rebound back to previous levels by day 30 post HSCT (FIG. 2C).

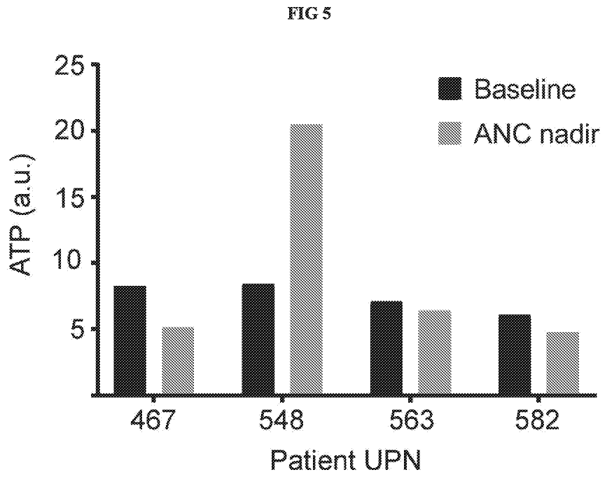

Gelsolin also participates in actin scavenging by VDBP, serving to de-polymerize F-actin to monomeric G-actin. We measured plasma gelsolin levels on day 7 after transplant, and found that levels above the median were associated with increased risk of TA-TMA (53% vs 32%, p=0.03), but gelsolin levels were not associated with any other outcome. We also hypothesized that endothelial toxicity may also be related to release of ATP into the circulation together with F-actin, but found no evidence of an increase in circulating ATP after HSCT. Extracellular ATP increased in only one of four HSCT recipients, in contrast to our hypothesis that extracellular ATP would increase markedly in most cases at the time of ANC nadir (time of maximum cell lysis) after HSCT (FIG. 5).

### VDBP Functions as a Macrophage-Activation Factor

Our findings regarding clearance of F-actin by VDBP provide a plausible mechanism for a role for VDBP in protection against endothelial injury and early toxicities of transplant such as TA-TMA and GVHD. However, As VDBP can also function as a macrophage activating factor, we investigated whether higher levels of VDBP functioned to modify cytokine production by immune cells later after transplant. Peripheral blood mononuclear cells from a normal healthy volunteer were incubated overnight with serum collected from 18 different HSCT recipients at four different time points, days 0, 30, 60 and 100 after HSCT. Cytokine levels in culture supernatants were measured by ELISA and cytokine concentrations were correlated with serum VDBP, total 25-hydroxy vitamin D (25-OH Vit D) and free vitamin D (free Vit D) (Table 4).

TABLE 4
Correlation of VDBP, total 25-hydroxy vitamin D (25-OH Vit D) and free vitamin D (free Vit D) with
interleukin-6 (IL6), tumor necrosis factor alpha (TNFα) and interleukin-10 (IL10) cytokine levels

| | | IL6 | | TNFα | | IL10 | |
|---|---|---|---|---|---|---|---|
| | | r² | p-value | r² | p-value | r² | p-value |
| Day 0 | VDBP | 0.14 | 0.58 | 0.079 | 0.75 | 0.16 | 0.52 |
| | 25-OH Vit D | 0.063 | 0.80 | 0.21 | 0.41 | 0.20 | 0.43 |
| | Free Vit D | 0.077 | 0.76 | 0.046 | 0.85 | −0.061 | 0.81 |
| Day 30 | VDBP | −0.07 | 0.83 | −0.27 | 0.37 | 0.10 | 0.73 |
| | 25-OH Vit D | −0.32 | 0.28 | −0.11 | 0.71 | 0.36 | 0.23 |
| | Free Vit D | −0.16 | 0.60 | 0.11 | 0.72 | 0.21 | 0.48 |
| Day 60 | VDBP | 0.53 | 0.028 | 0.52 | 0.027 | −0.65 | 0.0036 |
| | 25-OH Vit D | 0.19 | 0.45 | 0.23 | 0.35 | −0.30 | 0.22 |
| | Free Vit D | 0.18 | 0.18 | 0.12 | 0.64 | −0.16 | 0.53 |
| Day 100 | VDBP | 0.7 | 0.0013 | 0.28 | 0.27 | −0.46 | 0.05 |
| | 25-OH Vit D | 0.47 | 0.05 | −0.12 | 0.63 | −0.40 | 0.10 |
| | Free Vit D | −0.28 | 0.26 | −0.63 | 0.005 | 0.24 | 0.33 |

Data show no significant correlation between VDBP, total or free 25-hydroxy vitamin D and any of the three cytokines assayed at days 0 and 30. In contrast, the day 60 data show a significant positive association between VDBP and IL-6 and TNFα, and a significant negative association between VDBP and IL-10. The association between VDBP and IL-6 and IL-10 was also seen with sera collected at day 100. A positive association between 25-hydroxy vitamin D and IL-6 and a negative association between free 25-hydroxy vitamin D and TNFα was also observed at day 100.

These data suggest that serum VDBP and its cargo, 25-hydroxy vitamin D, can modify immune cell function, but this effect was only evident at later time points following transplant. In order to better understand the nature of this immune modulation we incubated peripheral blood mononuclear cells from a normal volunteer overnight with serum from (i) an HSCT recipient who had stable levels of VDBP at baseline and day 60 and (ii) an HSCT recipient in whom VDBP levels increased significantly at day 60 compared with baseline. Changes in gene expression profile between the same cells conditioned with serum from these 2 distinct patients were compared using RNAseq, and the genes with the most notable change in expression are listed in Table 5.

TABLE 5
Genes of PBMC from normal healthy donor having the most highly
dysregulated expression after incubation with serum collected from
transplant recipients 100 days after HSCT compared with serum
collected at day 0.

| | | Change |
|---|---|---|
| | | with |
| | | increased |
| Gene | Function | DBP |
| SIGLEC-1 | Macrophage marker, induced by TLR | Increased |
| (CD169) | agonists [18] | 8.3 fold |
| ADORA3 | Adenosine A1 receptor; macrophage gene | Increased |
| | that regulates IL6 and TNF production | 8.2 fold |
| | and suppress IL12 [19] | |
| IL1R2 | Interleukin 1 receptor, type II; expression | Increased |
| | increased in M2 macrophages [20] | 7.7 fold |
| SH3PXD2B | LPS suppresses the phagocytic function of | Increased |
| | macrophages by down-regulation of | 7.2 fold |
| | SH3PXD2B expression through TLR4 | |
| | pathway [21] | |
| TMIGD3 | Encodes a transmembrane and immuno- | Increased |
| | globulin domain-containing protein that has | 6.5 fold |
| | overlapping activities with ADORA3 [22] | |
| IFI44L | IFN1 signature gene [23] | Increased |
| | | 6.4 fold |
| SERPINB2 | M2 “specific” marker [24] | Increased |
| | | 6.2 fold |
| IFIT3 | Part of IFN1 signature, induced by and | Increased |
| | active against viruses [23] | 6.2 fold |
| C1QB | Marker of macrophage activation [25] | Increased |
| | | 6.1 fold |
| IFI6 | IFN1 signature gene [23] | Increased |
| | | 5.3 fold |
| MARCO | Macrophages need MARCO to bind bac- | Increased |
| (macro- | teria and this activity is impaired by free | 4.8 fold |
| phage | actin. Actin scavenging has been linked | |
| receptor | to MARCO activity. [25, 26] | |
| with | | |
| collagenous | | |
| structure) | | |
| IL6 | Produced by macrophages after stimulation | Increased |
| | by the anti-inflammatory cytokine IL-4. [25] | 4.6 fold |
| CCL22 | Expression increased in M2 macrophages | Reduced |
| | [27] | 0.6 fold |
| CCL5 | Expressed in M1 macrophages [28] | Reduced |
| | | 0.44 fold |
| CLEC4A | Expression increased in M2 macrophages | Reduced |
| | [29] | 0.28 fold |
| MMP9 | Expression increased in M2 macrophages | Reduced |
| | [29] | 0.1 fold |

Observed changes in gene expression were consistent with altered macrophage function, consistent with the established potential for a deglycosylated form of VDBP to act as a macrophage activating factor. The RNAseq data, together with the change in cytokine profile, supported a possible change in macrophage polarization as a consequence of exposure to VDBP after HSCT.

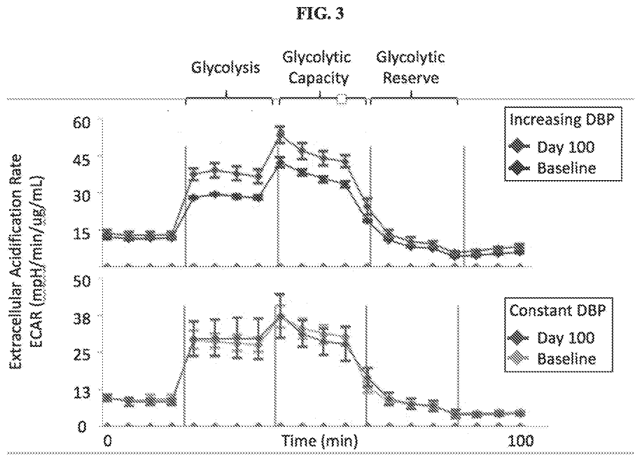

We addressed this further by incubating CD14/16 expressing normal peripheral blood macrophages separately with serum collected from the same HSCT recipient who exhibited increasing levels of VDBP from a baseline VDBP level of 78 to a day 100 VDBP level of 907 (genotype Gc1F/Gc1F) and also from a second HSCT recipient in whom VDBP levels were more constant (baseline VDBP level 681, Day 100 level 727, genotype Gc1S/Gc2). We then assessed macrophage polarization in the resulting cell cultures by measuring the extracellular acidification rate (ECAR) during a glucose stress test performed using Seahorse technology (FIG. 3). Data indicate altered macrophage polarization in cells incubated with serum containing a higher level of VDBP at day 100 (FIG. 3, top panel), but not in cells incubated with serum containing lower levels of VDBP at day 100 (FIG. 3, bottom panel).

VDBP macrophage activating activity has been reported to vary by genotype and by post-translational glycosylation. We hypothesized that changes in glycosylation of VDBP during the course of transplant might play a role in modifying macrophage polarization after HSCT. We tested this hypothesis by measuring glycosylation of VDBP in serum samples from two persons of differing VDBP genotype, collected at days 0 and 100 after HSCT (four samples total) to see if there were significant changes in glycosylation during recovery after HSCT. The only significant change in glycosylation between days 0 and 100 occurred at a single minor glycan (HexNAc(1)Hex(1)NeuAc(2)), with no significant change seen in the major glycosylated glycan (HexNAc(1)Hex(1)NeuAc(1)), suggesting that changes in glycosylation are likely not a major contributor to changes in immune modulation by VDBP.

### Discussion

Endothelial injury immediately following HSCT is an important initiator of later complications such as TA-TMA and GVHD, which together are major causes of morbidity and mortality after HSCT. Effector mechanisms leading to damage to the endothelium have not been described. In this study we show that release of the angiopathic molecule F-actin into the circulation is associated with TA-TMA, an immediate clinical consequence of endothelial damage. F-actin is typically not present in the circulation of healthy individuals who have low and relatively constant levels of cell turnover that do not exceed the capacity of the actin scavenger system for rapid removal of the harmful protein. Rapid lysis of the entire hematopoietic system during conditioning therapy for HSCT overwhelms actin scavenging, at least in some cases, and allows F-actin to remain in the circulation and cause damage. Similar findings have been described in the settings of acute liver failure and massive trauma, and low VDBP levels predict poor outcome in both these clinical settings, in agreement with our findings in this study (Schiodt et al., Crit Care Med, 1997. 25(8):1366-70; Dahl et al., Crit Care Med, 1998. 26(2):285-9; Lee et al., Hepatology, 1995. 21(1):101-5; Goldschmidt-Clermont, Lee, and Galbraith, Gastroenterology, 1988. 94(6):1454-8; Dahl et al., Injury, 1999. 30(4):275-81. Our findings differ from reports of acute liver failure in that HSCT recipients generally have healthy livers and we were able to see rapid rebound of VDBP levels after initial depletion.

In this paper we show for the first time that VDBP levels influence outcomes of HSCT. Our data show both an association between day 30 VDBP level and TA-TMA, and a strong association of day 100 level with NRM. Our data showed a differential effect of VDBP genotype on the early outcome of TA-TMA, with lower TA-TMA in Gc2 homozygous transplant recipients. A large number of prior epidemiological studies have identified associations between VDBP polymorphism and assorted chronic and frequent diseases, including chronic obstructive lung disease, diabetes mellitus and coronary artery disease, supporting some functional importance to the variants, although reproducibility of association studies is mixed. Despite the many association studies, there have been few functional studies that offer insight into potential mechanisms. Gc1 and Gc2 alleles differ by a single amino acid and by differential post-translational glycosylation. Gc1s and Gc1f alleles differ by a single amino acid but are similarly glycosylated. The functional consequences of these changes are poorly understood, although in general Gc2 homozygotes do have modestly reduced VDBP levels.

VDBP functions as an actin scavenger, and in this role has been shown to be key in recovery from massive cell lysis occurring in settings such as acute liver failure and massive trauma. Higher levels of VDBP are associated with improved survival in those clinical settings, likely because VDBP serves to remove the angiopathic molecule F-actin, similar to the findings in our study. F-actin is clearly identified as directly angiopathic in previous literature. Surprisingly, however, direct infusion of monomeric actin into dbp null mice showed increased pulmonary toxicity in the dbp deficient compared to dbp sufficient animals. This study raises a concern that the angiopathic molecule might be the actin-VDBP complex, and not unbound free actin. This distinction is important because if the actin-VDBP complex is the toxic molecule, rather than unbound free actin, then infusion of exogenous VDBP to an HSCT recipient near the time of transplantation could be fatal, instead of beneficial. However, the data presented here indicate that there is a significant difference between human and mouse, and that in humans higher serum VDBP beneficial, and is associated with better outcomes. In contrast to the earlier murine studies, our work indicates that VDBP could be prepared and used therapeutically to reduce the risk of microangiopathy, graft versus host disease, and non-relapse mortality. For example, since VDBP is a very abundant protein in serum, it could be produced using techniques similar to those used to produce human serum albumin, and administered to HSCT recipients, for example by infusion.

We found considerable variation in the amount of actin-VDBP complex present at day zero (the day of stem cell infusion, after completion of conditioning therapy), between individuals. The amount of complex present at day zero could be influenced by many variables, including the amount of hematopoiesis present to be lysed (high in active leukemia, low in marrow failure), the VDBP level, the efficiency of the reticulo-endothelial system in removing complex and of the liver in replacing VDBP and the intensity and speed of delivery of the preparative regimen so variability is perhaps not surprising.

The benefit of VDBP appears to last longer than the early weeks after transplant when endothelial injury occurs, and in this context appear independent of vitamin D transport. A higher day 100 VDBP level was associated with significantly improved NRM, a time long after lysis of the host hematopoietic system and clearance of F-actin, but a critical time for recovery of the donor immune system and for ongoing healing of epithelial injury. VDBP is known to act as a so-called “macrophage activating” factor, so we hypothesized that VDBP levels might favorably influence immune recovery and tissue healing in the later weeks after HSCT. We found changes in cytokine secretion and changes in expression of genes related to macrophage polarization associated with VDBP levels. For example, marked up-regulation of ADORA3 would promote IL6 production which would favor epithelial healing. We propose that higher levels of VDBP at later time points serve to modulate immune recovery, while at early time points, prior to donor cell recovery, there are few if any macrophages to modulate so no effect is apparent.

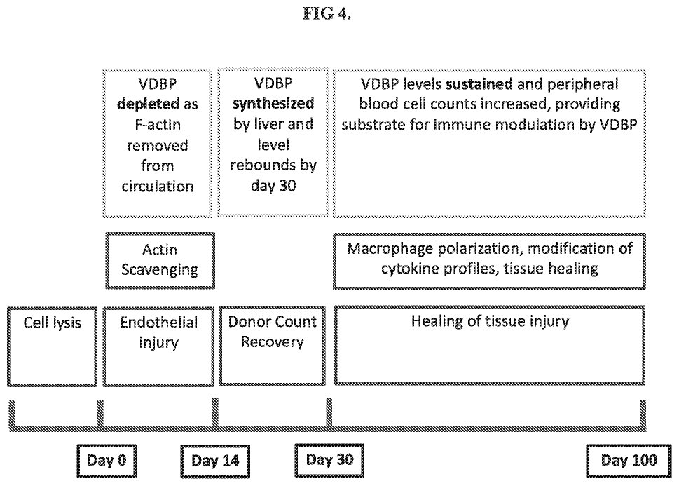

FIG. 4 shows in schematic form our proposal for VDBP function after HSCT. We envisage that in the early days after transplant VDBP functions as an actin scavenger, protecting the endothelium and preventing early complications such as TA-TMA. VDBP is depleted in this process, but rebounds around day 30. VDBP has little opportunity to act as an immunomodulator in the early weeks after HSCT as donor cell counts have not yet recovered. Between days 30 and 60 after HSCT donor cell counts recover and tissue healing takes place, and VDBP can modify macrophage phenotype to favor tissue healing. Taken together, these data indicate important benefit for higher levels of VDBP after HSCT, and further work will clarify whether there would be potential benefit from strategies to increase or infuse additional VDBP.

### Methods.

### Patients

Patient samples were obtained from the Cincinnati Children's Hospital Medical Center (CCHMC) HSCT repository. All children and families receiving HSCT at CCHMC are offered enrollment in the CCHMC HSCT sample repository, approved by the CCHMC IRB. Weekly blood, urine and stool samples are collected from consented patients, starting prior to the start of conditioning therapy and continuing past day 100. Demographics of the 190 patients included in the study are shown in Table 1. The focus of this study is endothelial injury so careful attention was given to prospective phenotyping for TA-TMA including monitoring for schistocytes, LDH, haptoglobin, proteinuria, elevation of creatinine, new and excessive thrombocytopenia and anemia, as previously reported (Jodele et al., Blood, 2014. 124(4):645-53).

### Analysis of Serum Vitamin D Metabolites by Ultra-High-Performance Liquid Chromatography

Serum samples were collected prospectively on consenting HSCT receiving their first HSCT, and samples were stored at −80° C. until analyzed. Human serum concentrations of 25-hydroxyvitamin D2 (25-OH D2) and 25-hydroxyvitamin D3 (25-OH-D3) were determined by ultra-high-performance liquid chromatography coupled to electrospray tandem mass spectrometry (Waters, Milford, Mass.). Serum samples were extracted by liquid-liquid extraction with methyl tert-butyl ether/ethyl acetate/hexane (5:4:1). Combined extracts were dried and derived by 4-phenyl-1,2,4-thriazoline-3,5-dione before transfer to sample vials. Quantification was conducted with multiple reaction monitoring and with a stable isotope dilution ultra-high-performance liquid chromatography coupled to electrospray tandem mass spectrometry method on a Supelcosil LC-18-DB column (33-3 mm, 3 mm; Sigma, St. Louis, Mo.). A vitamin D level<30 ng/mL was defined as vitamin D insufficiency, and a vitamin D level<20 ng/mL was defined as vitamin D deficiency.

### ELISA Testing

Serum VDBP levels were measured using a polyclonal antibody ELISA (Genway Bio, DM3741), and commercial ELISA kits were used to measure serum F-actin (MyBioSource, 702018), IL6 (R&D Systems Quantikine, D6050), TNFα (R&D Systems Quantikine, DTA00C), IL10 (R&D Systems Quantikine, D1000B), and gelsolin (LSBio, F22526).

### Calculation of Bioavailable 25-Hydroxyvitamin D

Free vitamin D is defined as circulating 25-OH D not bound to VDBP, but does include the albumin bound fraction. Free vitamin D levels were calculated from measurements of total serum 25-OH-D2, 25-OH-D3, and DBP using previously documented equations that incorporate both genotype-specific and genotype-nonspecific affinity constants (Chun, R. F., Cell Biochem Funct, 2012. 30(6):445-56).

### VDBP Genotyping

DNA samples from participants were genotyped for two common single-nucleotide polymorphisms (SNPs) in the coding region of the VDBP gene (Applied Biosystems, rs4588 and rs7041). TaqMan SNP Genotyping Assay protocol was performed following manufacture's guidelines using TaqMan Genotyping Master Mix and primer sets (Applied Biosystems, 4371355).

### Western Blot Analysis

Serum proteins were electrophoresed under native conditions through a precast 10% polyacrylamide gel (Bio-Rad, #456-1034) and transferred using conventional techniques. The PVDF membrane was probed with a mouse monoclonal antibody against native full-length, human plasma derived, VDBP (1:4000, Abcam, ab23480), then incubated with an HRP conjugated, anti-mouse IgG antibody (1:5000, Cell Signalling, 7076P2). Results were visualized by chemiluminescence (Amersham, RPN2235) using a BioRad ChemiDoc Touch imager. A positive DBP-actin complex band (˜250 kDa) was scored visually.

### Measurement of ATP Levels by Quantitative Mass Spectrometry

Samples were prepared from 10 uL of plasma by precipitation of proteins with a 3 volume cold acetone followed by centrifugation and injection of 15 uL of supernatant per sample. ATP quantitation was performed by LC-MS-SRM using a Shodex HILICpak VN-50 2D column, 5 μm, 2.1 mm×150 mm column, (New York, N.Y.) on a Vanquish™ Flex Quaternary UHPLC system (Thermo Fisher Scientific, San Jose, Calif.) coupled to a Thermo Scientific™ Quantiva™ triple quadrupole mass spectrometer by negative ion mode electrospray ionization and selected reaction monitoring (SRM) mass spectrometry using diagnostic transitions for ATP. A stock solutions of ATP was prepared and infused into the QQQ mass spectrometer for optimization of precursor transition as well as product ion fragmentation. To enhance detection specificity for ATP, the precursor at m/z 505.9 was monitored as two SRM transition fragment ions at 159 and 408 using collision energies of 2.9 and 17 volts, respectively.

The separation Mobile phase A consisted of acetonitrile (Honeywell Burdick & Jackson, Morris Plains, N.J.) Mobile phase B consisted of a 50 mM ammonium bicarbonate and with a gradient of 15% B (from 0 to 4 min) increasing to 35% B at 6 min, 75% B at 6.5 min (hold for 1 min), 15% B at 7.6 min, holding for 12.4 min for equilibration at a flow rate of 300 μL min⁻¹. The column temperature was set at 60 C. ATP signal intensity (arbitrary units) was monitored across the LC-MS-SRM run and the peak areas for the specific SRM transitions were captured versus retention time. The quantitative data are reported as peak areas of the SRM intensities across the chromatographic run.

### Cytokine Studies

Peripheral blood mononuclear cells (PBMC) from a normal individual were isolated by layering over Ficoll, then placed in a 24 well plates and cultured overnight. Culture medium included 10% serum from 18 different children, collected at days 0, 30, 60 and 100 after transplant (4 samples per child, 72 total wells). Supernatant was collected after overnight incubation and analyzed for concentration of IL6, TNFα and IL10 by ELISA as described above.

### RNAseq Studies

RNAseq studies were performed as described previously (Jodele et al., Blood 2016. 127(8):989-96). Briefly, normal PBMC were incubated overnight with sera from 2 HSCT recipients, collected at days 0 and 100 (4 wells total). RNA was extracted and submitted for RNAseq analysis. Gene expression at day 100 was compared with expression at day 0.

### Glucose Stress Test (Seahorse Analysis)

Macrophage polarization was examined by measurement of extracellular acidification rate (ECAR) using Seahorse technology. Briefly, normal PBMC were incubated overnight with sera collected at days 0 and 100 from 2 HSCT recipients. Cells were harvested and re-plated before performance of a glucose stress test and metabolic analysis.

### VDBP Glycosylation Studies

Protein concentration of each sample was determined by Qubit fluorometry. 10 μL of protein from each sample was depleted in duplicate on a Pierce Top 12 depletion spin cartridge according to manufacturer's protocol. Depleted samples were buffer exchanged into water on a Corning Spin×5 kD molecular weight cut off spin column and quantified by Qubit fluorometry (Life Technologies). 50 μg of each sample was reduced with dithiothreitol, alkylated with iodoacetamide and digested overnight with trypsin (Promega); digestion was terminated with formic acid. Each digested sample was processed by solid phase extraction using an Empore C18 (3M) plate under vacuum (5 in Hg) with the following protocol: Columns were activated with 400 μL 95% acetonitrile/0.1% TFA X2 and equilibrated with 400 μL 0.1% TFA X4. Acidified samples were loaded and columns washed with 400 μL 0.1% TFA X2. Peptides were eluted with 200 μL 70% acetonitrile/0.1% TFA X2 and lyophilized for further processing. Half of each gel digest was analyzed by nano LC-MS/MS with a Waters NanoAcquity HPLC system interfaced to a ThermoFisher Q Exactive. Peptides were loaded on a trapping column and eluted over a 75 μm analytical column at 350 nL/min with a 1 hr reverse phase gradient; both columns were packed with Luna C18 resin (Phenomenex). The mass spectrometer was operated in data-dependent mode, with the Orbitrap operating at 60,000 FWHM and 17,500 FWHM for MS and MS/MS respectively. The fifteen most abundant ions were selected for MS/MS.

### Statistics

Categorical and continuous demographics are described by frequency (percent) and median (range), respectively. Time to event data, NRM, GVHD and TMA are described at 1 year and with cumulative incidence curves using the Kaplan-Meier method incorporating death as a competing risk. In both the Day 30 and 100 analysis patients with an event preceding the date of measurement were excluded. This was done to ensure that the measured VDBP preceded the respective NRM, GVHD or TMA event. Gray's method for competing risks was used to test for differences in time to event between groups. Glycan data are summarized by mean and levels were compared between day 0 and day 100 using a paired t-test.

Those skilled in the art will recognize or be able to ascertain using no more than routine experimentation, many equivalents to the specific embodiments of the invention as described herein. Such equivalents are intended to be encompassed by the following claims.

All references cited herein are incorporated herein by reference in their entirety and for all purposes to the same extent as if each individual publication or patent or patent application was specifically and individually indicated to be incorporated by reference in its entirety for all purposes.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

1. A method for identifying a hematopoietic stem cell transplant (HSCT) recipient at increased risk for post-HSCT non-relapse mortality or a transplantation associated adverse event, the method comprising determining ex vivo or in vitro a level of vitamin D binding protein (VDBP) in a serum or plasma sample obtained from the HSCT recipient, wherein a level of VDBP below a predetermined threshold identifies the HSCT recipient as being at increased risk of non-relapse mortality or a transplantation associated adverse event.

2. The method of claim 1, wherein the method comprises determining ex vivo or in vitro the level of one or more additional proteins in the serum or plasma sample of the HSCT recipient, the one or more additional proteins selected from F actin, IL-6, IL-10, and TNFα.

3. The method of claim 1, wherein the step of determining the level of VDBP or the level of the one or more additional proteins in the serum or plasma sample comprises an immunoasssay, optionally an enzyme linked immunosorbent assay (ELISA).

4. The method of claim 1, wherein the transplant associated adverse event is selected from transplant-associated thrombotic microangiopathy (TA-TMA) and graft versus host disease (GVHD).

5. A method for monitoring a therapy of an HSCT recipient, the method comprising determining ex vivo or in vitro a level of VDBP in a serum or plasma sample obtained from the HSCT recipient, wherein a level of VDBP below a predetermined threshold indicates that the recipient is at increased risk of non-relapse mortality or a transplantation associated adverse event and that the therapy should be adjusted.

6. The method of claim 5, wherein the method comprises determining the level of VDBP at multiple time points during the therapy.

7. The method of claim 6, further comprising determining whether the level of VDBP is increasing or decreasing over time.

8. A method of treating an HSCT recipient, the method comprising administering purified or recombinant human VDBP to the recipient following transplantation.

9. The method of claim 1, wherein the recipient is human.

10. The method of claim 9, wherein the HSCT recipient has been diagnosed with a lymphoma, leukemia, immune-deficiency disease or disorder, a congenital metabolic defect, a hemoglobinopathy, a myelodysplastic syndrome, or a myeloproliferative syndrome.

11. The method of claim 9, wherein the HSCT recipient has been diagnosed with a hematological malignancy or premalignant condition.

12. The method of claim 11, wherein the hematological malignancy or premalignant condition is selected from acute myeloid leukemia (AML), acute lymphoblastic leukemia, chronic myeloid leukemia, Hodgkin lymphoma (HL), non-Hodgkin lymphoma (NHL), and multiple myeloma.

13-16. (canceled)
